(19) United States
(12) Patent Application Publication
(10) Pub. No.: US 2011/0054049 A1
(43) Pub. Date: Mar. 3, 2011

(54) OXIDATION-REDUCTION ACTIVE MASS AND CHEMICAL-LOOPING COMBUSTION METHOD

(76) Inventors: Arnold Lambert, Chavanay (FR); Thierry Gauthier, Brignais (FR)

(21) Appl. No.: 12/990,383

(22) PCT Filed: Apr. 29, 2009
(86) PCT No.: PCT/FR2009/000511

§ 371 Date: Oct. 29, 2010

(30) Foreign Application Priority Data
Apr. 30, 2008 (FR) 0802449

(51) Int. Cl. C07C 1/04; C01B 3/30; C01B 31/20; C01B 3/38; F02C 1/00

(52) U.S. Cl. 518/719; 423/651; 423/437.1; 252/373; 60/772

(57) ABSTRACT

The invention relates to a method for chemical-looping redox combustion on an active mass including a binder, in form of a fluidized-bed catalytic cracking catalyst containing silica and alumina, and a metal oxide active phase. The active mass is obtained by impregnating metal salts on a new or used catalytic cracking catalyst. Advantageously, the invention applies to the sphere of CO₂ capture.

### FIELD OF THE INVENTION

The invention relates to an active mass and to a fluidized-bed CO₂ capture method using this active mass.

We have discovered that a catalyst that has been used in fluidized-bed catalytic cracking plants, generally considered to be valueless industrial waste and usually incorporated in cement or road asphalts, can be used, after impregnation of a metal salt, in a redox loop combustion method allowing notably CO₂ sequestration.

The invention consists in using silica and alumina based catalysts shaped to have facilitated flow and transport properties, such as the catalysts used in catalytic cracking processes, in impregnating these catalysts with metal salt solutions, preferably based on iron, nickel, copper, cobalt or manganese, and in using these impregnated catalysts in a combustion process consisting of a zone wherein the fuel is oxidized by the oxygen supplied through reduction of the impregnated catalyst, and of a zone of oxidation of the impregnated catalyst in the presence of air, the impregnated catalyst circulating on a continuous basis between these two reduction and oxidation zones.

The method allows to carry out combustion of the fuel and notably to produce nitrogen-free and CO₂-concentrated fumes that facilitate CO₂ sequestration. The impregnated catalyst according to the invention (active mass) is particularly interesting because it has physical properties that facilitate its circulation (diameter, density) and mechanical properties such as attrition resistance that facilitate its use in such a process.

### TERMINOLOGY

Catalytic cracking (FCC: Fluid Catalytic Cracking): What is referred to as catalytic cracking is a method of converting heavy petroleum fractions, without hydrogen supply, using a high temperature (generally of the order of 500° C. to 600° C.) and a cracking catalyst (generally a solid of acidic character such as a silica and alumina based solid or a zeolite). The catalyst comes in form of a fine powder (approximately 50 to 100 μm mean diameter) that circulates in fluidized state in the plant (fluidized-bed catalytic cracking). The heavy feeds are, for example, vacuum distillates, deasphalted oils or more or less severely hydrotreated petroleum residues, which are converted to light fractions (LPG, gasoline) by means of the catalytic cracking method.

Chemical Looping Combustion method or CLC: In the text hereafter, what is referred to as CLC (Chemical Looping Combustion) is an oxidation-reduction or redox looping method using an active mass. It can be noted that, in general, the terms oxidation and reduction are used in connection with the respectively oxidized or reduced state of the active mass. The oxidation reactor is the reactor where the redox mass is oxidized and the reduction reactor is the reactor where the redox mass is reduced.

### BACKGROUND OF THE INVENTION

In a context of increasing world energy demand, capture of carbon dioxide for sequestration thereof has become an imperative necessity in order to limit greenhouse gas emissions harmful to the environment. The Chemical Looping Combustion (CLC) method allows to produce energy from hydrocarbon-containing fuels while facilitating capture of the carbon dioxide emitted during the combustion.

The CLC method consists in using redox reactions of an active mass so as to split the combustion reaction into two successive reactions. A first reaction of oxidation of the active mass, with air or a gas acting as the oxidizer, allows the active mass to be oxidized.

A second reaction of reduction of the active mass thus oxidized, using a reducing gas, then allows to obtain a reusable active mass and a gaseous mixture essentially comprising carbon dioxide and water, or even synthesis gas containing hydrogen and nitrogen monoxide. This technique thus allows to isolate the carbon dioxide or the synthesis gas in a gaseous mixture practically free of oxygen and nitrogen.

The combustion being globally exothermic, it is possible to produce energy from this method, in form of vapour or electricity, by arranging exchange surfaces in the active mass circulation loop or on the gaseous effluents downstream from the combustion or oxidation reactions.

U.S. Pat. No. 5,447,024 describes a CLC method comprising an active mass reduction reactor using a reducing gas and an oxidation reactor allowing the active mass to be restored in its oxidized state by means of an oxidation reaction with wet air. The circulating fluidized-bed technology is used to allow continuous change of the active mass from its oxidized state to its reduced state.

The active mass that alternately changes from its oxidized form to its reduced form, and vice versa, follows a redox cycle.

Thus, in the reduction reactor, the active mass (MₓO_{y}) is first reduced to the state MₓO_{y-2n-m/2}, by means of a hydrocarbon which is correlatively oxidized to CO₂ and H₂O, according to reaction (1), or possibly to mixture CO+H₂ depending on the proportions used.

CₙHₘ+MₓO_{y} [symbol] nCO₂+m/2H₂O+MₓO_{y-2n-m/2} (1)

In the oxidation reactor, the active mass is restored to its oxidized state (MₓO_{y}) on contact with air according to reaction (2), before returning to the first reactor.

MₓO_{y-2n-m/2}+(n+m/4)O₂ [symbol] MₓO_{y} (2)

The efficiency of the circulating fluidized bed CLC method is based to a large extent on the physico-chemical properties of the redox active mass.

The reactivity of the redox pair(s) involved and the associated oxygen transfer capacity are parameters that influence the dimensioning of the reactors and the rates of circulation of the particles.

The life of the particles depends on the mechanical strength of the particles and on their chemical stability.

In order to obtain particles usable for this method, the particles involved generally consist of a redox pair or a series of redox pairs selected from among CuO/Cu, Cu₂O/Cu, NiO/Ni, Fe₂O₃/Fe₃O₄, FeO/Fe, Fe₃O₄/FeO, MnO₂/Mn₂O₃, Mn₂O₃/Mn₃O₄, Mn₃O₄/MnO, MnO/Mn, Co₃O₄/CoO, CoO/Co, and of a binder providing the required physico-chemical stability.

U.S. Pat. No. 5,447,024 claims as the active mass the use of the redox pair NiO/Ni, alone or combined with the binder YSZ (yttrium-stabilized zirconia, also referred to as yttriated zirconia). In addition to the improved mechanical strength of the particles, the yttriated zirconia being an ionic conductor of O²⁻ ions at the operating temperatures used, the reactivity of the NiO/Ni/YSZ system is also improved.

Many binder types, aside from the aforementioned yttriated zirconia (YSZ), have been studied in the literature in order to increase the mechanical strength of the particles at a lower cost than YSZ. Examples thereof are alumina, metal aluminate spinels, titanium dioxide, silica, zirconia, kaolin.

The redox pair/binder mass ratio is generally around 60/40 in order to obtain particles having a good mechanical strength, as well as sufficient redox properties (oxidation and reduction rate, oxygen transfer capacity).

Document EP-1,747,813 describes redox masses comprising a redox pair or a set of redox pairs selected from the group made up of CuO/Cu, Cu₂O/Cu, NiO/Ni, Fe₂O₃/Fe₃O₄, FeO/Fe, Fe₃O₄/FeO, MnO₂/Mn₂O₃, Mn₂O₃/Mn₃O₄, Mn₃O₄/MnO, MnO/Mn, Co₃O₄/CoO, CoO/Co, in combination with a ceria-zirconia type binder allowing the oxygen transfer capacity of said masses to be increased.

Using a binder allows to provide the mechanical strength required for the particles, but it also increases the cost price of the particles involved in the CLC method.

It is therefore important to find an optimized catalyst for the looping redox method using an active mass, at a lesser cost. In order to decrease the impact of the cost of the particles on the cost of the CO₂ capture by CLC, the Instituto de Catalisis y Petroleoquimica (CSIC) in Madrid has shown that it is possible to use an ilmenite ore (FeTiO₃) (“Titania-supported iron oxide as oxygen carrier for chemical-looping combustion of methane, Corbella, Beatriz M.; Palacios, Jose Maria, Fuel (2006), Volume Date 2007, 86(1-2), 113-122).

We have discovered that using waste from the refining industry, in form of used catalytic cracking catalysts, as a binder in combination with one or more metal oxides, allows to obtain an active mass for CLC at a cost price that is all the lower as the geometrical and structural characteristics of the FCC catalyst particles are optimized for fluidization and attrition resistance, and as the amounts of used catalyst generated by the fluidized-bed catalytic cracking method are large.

In fact, catalytic cracking is a method that is used worldwide to convert heavy petroleum fractions such as vacuum distillates, deasphalted oils or more or less severely hydrotreated petroleum residues, to light fractions (LPG, gasoline).

In the catalytic cracking method, the feed is converted using acid-catalyzed cracking reactions. The presence of metals on the catalyst is detrimental to the process. Therefore, in order to maintain an acceptable performance level, refiners regularly add fresh cracking catalyst (containing no metals) and withdraw a proportion of cracking catalyst referred to as <<used>> from the unit, in order to maintain a reasonable metal concentration on the catalyst.

Sizeable amounts of fresh catalyst are therefore added in the units to maintain a stable catalytic activity over time. The more the refiner treats metallized feeds, the more he will have to add catalyst in order to keep a reasonable metal content in the catalyst.

However, there is currently no noble use of this used catalyst. It is generally sent to cement works or used as a coating agent for road pavement coatings. On a world scale, this material however represents very large amounts. Several hundred thousand tons are annually available on a world scale, at a marginal cost since the used catalyst has no noble use and can therefore be considered as waste.

The used catalysts in catalytic cracking units contain, after use, between 50 and 20,000 ppm metals that decrease their catalytic activity for this process, in particular Ni and V. Such a content is not sufficient to consider using these materials, currently considered to be waste, in an energy production plant by means of the loop redox method on active mass, because the solid circulation rates required for combustion would be too high. However, after impregnation of the catalyst by one or more metal salts and calcination, the particles obtained have interesting characteristics for use in a circulating fluidized bed:

grain size suited to fluidization,

redox properties,

attrition resistance.

### OBJECTS OF THE INVENTION

The invention relates to an active mass for combustion comprising a binder, in form of a fluidized-bed catalytic cracking catalyst (new or used) based on silica and alumina, and a metal oxide active phase obtained by impregnation of metal salts on the catalyst.

The invention also relates to a looping redox combustion method using as the active mass said impregnated catalyst.

### DESCRIPTION OF THE INVENTION

The invention consists in taking a catalyst used for petroleum feed conversion, fresh or after use in a catalytic cracking unit, in impregnating it with one or more metal salts and in using it thereafter in a looping redox method on active mass of Chemical Looping type.

### SUMMARY OF THE INVENTION

The invention relates to a combustion method intended for solid, liquid or gaseous hydrocarbons through chemical looping oxidation-reduction using an active mass comprising at least one silica and alumina based binder in form of a fluidized-bed catalytic cracking catalyst and at least one metal oxide in a proportion ranging between 5 and 95 mass %.

Advantageously, the active mass is a used fluidized-bed catalytic cracking catalyst.

Preferably, the metal oxide content ranges between 20 and 70 mass %, more preferably between 30 and 60%.

Advantageously, the metal oxide is based on at least one element selected from among Co, Fe, Mn, Cu, Ni, and it is preferably based on Fe.

The combustion can be total or partial.

When the combustion is partial, the method allows to produce a synthesis gas (CO+H₂) that can constitute a gaseous feed usable in the Fischer-Tropsch liquid hydrocarbon synthesis process.

In this case, when the gas allowing fluidization of the active mass comprises steam, a gaseous mixture containing (CO₂+H₂) is produced at the outlet. The method can then be used to produce hydrogen.

The method according to the invention (partial or total combustion) can be used to produce energy.

The invention relates to a CO₂ capture method through total chemical looping combustion in a method according to the invention.

In the method according to the invention, the active mass can be prepared as follows:

a. a stage of impregnation by at least one metal salt of a fluidized-bed catalytic cracking catalyst,

b. a stage of drying and/or calcination of the impregnated catalyst,

The active mass can be:

b. dried,

c. injected after drying in the oxidation reactor.

The active mass can be:

b. calcined,

c. injected after calcination in the reduction oven.

### DETAILED DESCRIPTION OF THE INVENTION

### Description of the Catalytic Cracking Catalyst

In the catalytic cracking method, the feed is converted using acid-catalyzed cracking reactions. It is well known that the acidity of the catalyst can be obtained by using silica and alumina based solids, or complex crystal structures such as zeolites. The catalytic cracking catalyst generally comprises one or more zeolites.

During the reaction, coke forms, settles on the catalyst and leads to fast deactivation thereof. It is therefore necessary to carry out continuous regeneration of the catalyst.

The process thus generally consists of a reaction zone, wherein the feed encounters the catalyst under suitable conditions, and of a regeneration zone wherein the coke that has settled on the catalyst during the reaction is burned in the presence of oxygen. The catalyst circulates on a continuous basis between the reaction zone and the regeneration zone. After regeneration, the catalytic activity of the catalyst is restored through the combustion of the coke. The catalyst thus undergoes a succession of reaction-regeneration cycles.

In this method, the constraints linked with the regeneration of the catalyst and the thermal balance of the unit are such that the catalyst circulation is very high between the two enclosures. For a given feed flow rate, the catalyst circulation is generally close to 3 to 10 times the feed flow rate, generally 5 to 7 times, and for a unit treating around 40,000 BPD, which is the average capacity of the units currently in operation, the continuous catalyst circulation is typically about 1300-1800 t/h, the average travel time required for the catalyst to go round the unit generally ranging between 3 and 10 minutes.

Throughout the process, the catalyst is maintained in fluid state through control of the fluidization gas flow rates at all points of the unit. This is essential to ensure smooth running of the process, which can only be obtained if the catalyst particles are shaped with particular properties: it is essential that the particles belong to group A of Geldart's classification (Gelded D., Powder Technology, 7, p. 285-292 (1973)). Preferably, the mean diameter (Sauter) of the particles ranges between 50 and 100 microns, preferably around 70 microns, and the grain density ranges between 1000 and 3500, or even 5000 kg/m³ if the diameter of the particles tends towards 50 microns. Furthermore, the grain size distribution of the powder is preferably wide. This goal is reached by shaping the catalyst by means of techniques such as spray drying. Under such conditions, it is possible to produce powders whose mean diameter ranges between 50 and 100 microns, but which contain large amounts of fine particles (preferably 5 to 20 wt. %).

During fluidized-bed catalytic cracking, the catalyst undergoes a substantial deactivation between each reaction cycle and each regeneration cycle associated with the coke deposition. The type of feeds treated in this method generally contains metals, for example nickel, vanadium, iron (Ni, V, Fe) in small amounts. These metals, upon contact between the feed and the catalyst, settle on the catalyst and accumulate there progressively. During regeneration, carried out, depending on the technologies implemented, at temperatures ranging between 600° C. and 850° C., typically around 750° C., on contact with air and steam, the catalyst can undergo modifications, accentuated if the proportion of metal feed deposited on the catalyst is significant.

Generally, the feed to be treated in the catalytic cracking unit (Fluid Catalytic Cracking or FCC) contains 0 to 50 ppm, preferably 0 to 20 ppm nickel and vanadium (Ni+V). The iron content of the feeds is more occasional, but it may be high in the feeds treated. The metal concentration on the catalyst in operation naturally depends on the metal concentration in the feeds, which generally ranges between 0 and 20,000 ppm for nickel and vanadium, generally between 5 and 10,000 ppm. Occasionally, the concentration can be higher when constituents such as iron are encountered in the feeds to be converted.

The presence of metals on the catalyst is harmful to the operation of the process. The hydrothermal stability of the acidic catalyst (silica-alumina) is affected by the presence of metals, in particular vanadium. Furthermore, some metals such as nickel promote hydrocarbon dehydrogenation and therefore lead to higher coke yields. The combustion of coke is also sensitive to the deposition of metals on the catalyst. Under oxygen deficiency conditions, the CO/CO₂ ratio varies significantly depending on these deposits.

Assuming that all the metals of the feed settle on the catalyst, one can simply estimate the amount A of fresh catalyst to be added to keep a reasonable metal concentration CMc on the catalyst according to the metal concentration in the feed CMf and to the feed flow rate FE:

A=FF*CMf/CMc.

For a unit treating 6000 t/d of feed, if the metal content (Ni+V) of the feed is 4 ppm, 3 t/d of fresh catalyst have to be added to maintain a metal content of 8000 ppm on the catalyst. If the metal content is higher, for example 20 ppm Ni and V, a case that is frequently encountered in residue catalytic cracking units, the addition of 15 t/d of catalyst is then necessary.

In order to maintain a constant inventory in the unit, an equivalent amount of catalyst therefore has to be withdrawn from the unit, by taking account of catalyst entrainments with the products and fumes, which are however relatively low (generally of the order of 1 t/d). This catalyst withdrawn from the unit represents a substantial amount of catalyst that still has satisfactory flow properties and a high porosity (the specific surface area still is above 50 m²/g).

Advantageously, a fresh fluidized-bed catalytic cracking catalyst is made up of zeolite and of a matrix. The most commonly used zeolite is the USY zeolite. In some cases, other zeolites are used, such as ZSM-5, often as an additive, in a proportion of 1 to 15% in the inventory of the catalytic cracking unit, so as to confer particular properties on the catalyst and, for example, to maximize the propylene production. The zeolite content of the catalyst generally ranges between 10 and 50 wt. %. Preferably, a catalytic cracking catalyst comprises a USY or ZSM-E zeolite integrated in a silica-alumina matrix of variable composition.

The specific surface area of the fresh catalyst is generally around 300-350 m²/g with the USY zeolite and 150 m²/g with the ZSM-5. With the USY zeolite, the specific surface area developed by the matrix is around 30 to 150 m²/g, generally around 60 m²/g, and the specific surface area developed by the zeolite ranges between 150 and 300 m²/g, generally around 250 m²/g. In the used catalyst, these properties are modified. The specific surface area of the used catalyst is generally close to 100-180 m²/g with the USY zeolite. The specific surface area developed by the matrix is around 20 to 70 m²/g, typically 30 m²/g, and the specific surface area developed by the zeolite ranges between 50 and 150 m²/g, generally around 100-120 m²/g.

The Si/Al molecular ratio of the ultrastable zeolite varies from the new catalyst to the used catalyst. Thus, in a new catalyst, the Si/Al ratio of the zeolite is generally around 2 to 4. Due to the dealumination linked with the deactivation in the process, this ratio increases and it is generally around 4 to 10, often close to 5-6. Rare-earth oxides are sometimes incorporated into the catalyst to favour the hydrothermal stability of the zeolite, in a proportion of 0 to 5 wt. %.

Preparation of the Catalysts Usable in the CLC Method According to the Invention

The FCC catalysts from the catalytic cracking process contain between 0 and 20,000 ppm nickel and vanadium, and sometimes iron. Nickel and iron have interesting redox properties for use within the context of chemical looping combustion and they have been widely studied.

The metal content of the used FCC catalysts being very low, the direct use of these catalysts in the chemical looping combustion technology would require a high catalyst mass flow rate that is practically unthinkable in the current state of the circulating fluidized bed technology. In order to make these wastes from the refining industry useful for chemical looping combustion, the amount of metal contained in the used catalysts can be increased by impregnation of metal salts. The traces of nickel, iron and vanadium present in the used FCC catalyst contribute to the oxygen transfer capacity of the materials obtained after impregnation/calcination.

Similarly, a fresh FCC catalyst having, on account of its purpose, optimized grain size and attrition resistance for fluidization, it can be impregnated in order to obtain a redox active mass with a metal oxide(s) content in accordance with the invention.

The dry impregnation (or incipient wetness) method is advantageously used so as not to modify the initial size distribution of the particles, but any other impregnation type can be used, notably excess impregnation.

After impregnation, the particles can be either dried or calcined. Depending on the initial pore volume of the catalyst particles, on the concentration of the metal salt(s) solution and on the amount of oxide(s) to be deposited, the particles can undergo several successive impregnation/drying and/or impregnation/calcination cycles. The amount of impregnated metal salts is such that the particles contain between 5 and 95 mass % active metal oxide(s) (Ni, Cu, Fe, Co, Mn) after calcination between 600° C. and 1400° C., preferably between 20 and 80 mass %, and more preferably between 40 and 70 mass %. The matrix, made up of alumino-silicate and of zeolite, of the (fresh or used) FCC catalyst then acts as a binder for the metal oxide(s).

After calcination, the metal salts impregnated on the particles are in oxidized form. The calcination stage can optionally be carried out directly by feeding the impregnated FCC catalyst into the oxidation reactor, in which case the calcination effluents of the metal precursors used will be found at the outlet of said oxidation reactor.

Impregnation of the FCC catalyst is preferably performed by water-soluble metal precursors such as nitrates, sulfates, acetates, formates, halogenides or perchlorates. Metal salts soluble in organic solvents can also be used.

Description of the Fluidized Bed Co₂ Capture Method Using Chemical Looping Combustion

The active masses according to the invention act as oxygen carriers for the redox looping combustion method and they can be used for treating gaseous (natural gas, syngas), liquid (fuel oil, bitumen) or solid (coal) fuels in a circulating fluidized bed.

The impregnated catalyst is oxidized in a fluidized bed at a temperature ranging between 600° C. and 1400° C., preferably between 800° C. and 1000° C. It is then transferred to another fluidized-bed reactor where it is contacted with the fuel at a temperature ranging between 600° C. and 1400° C., preferably between 800° C. and 1000° C. The contact time typically ranges between 10 seconds and 10 minutes, preferably between 1 and 5 minutes. The ratio between the amount of solid active mass and the amount of feed to be burned ranges between 1 and 1000, preferably between 10 and 500.

The combustion can be partial or total.

In the case of partial combustion, the active mass/fuel ratio is adjusted so as to carry out partial combustion of the fuel, thus producing a synthesis gas in form of a CO+H₂ mixture.

The method can thus be used to produce a synthesis gas.

This synthesis gas can be used as a feed in other chemical conversion methods, the Fischer-Tropsch process for example, allowing to produce, from synthesis gas, liquid hydrocarbons with long hydrocarbon chains usable as fuel bases.

In cases where the fluidization gas used is steam or a mixture of steam and of other gas(es), the water-gas shift reaction (CO+H₂O [symbol] CO₂+H₂) can also take place, leading to the production of a CO₂+H₂ mixture at the reactor outlet.

In this case, the combustion gas can be used for energy production considering its calorific value.

It is also possible to consider using this gas for hydrogen production, for example in order to supply hydrogenation units, hydrotreatment units for refining, or a hydrogen supply network (after water-gas shift reaction).

In the case of total combustion, the gas stream at the reduction reactor outlet essentially consists of CO₂ and steam. A CO₂ stream ready to be sequestered is then obtained by condensation of the steam. Energy production is integrated to the Chemical Looping Combustion process by heat exchange in the reaction zone and on the fumes that are cooled.

The pressure of the method is adjusted according to the use of the combustion gases. Thus, to carry out total combustion, a low pressure is advantageously used to minimize the gas compression energy cost and thus to maximize the energy yield of the plant. To produce synthesis gas, one will advantageously work under pressure in some cases, in order to avoid compression of the synthesis gas upstream from the downstream synthesis process: the Fischer-Tropsch process operating for example at pressures ranging between 20 and 40 bars, it may be interesting to produce the gas at a higher pressure.

### DESCRIPTION OF THE FIGURES

FIGS. 1 to 7 illustrate the invention without limiting the scope thereof.

FIG. 1 represents the evolution of the grain size distribution between a fresh catalytic cracking catalyst (FIG. 1A) and the active mass obtained after impregnation of metal salts on said catalyst and calcination (FIG. 1B) (example 1),

FIG. 2 shows the evolution of the grain size distribution between a used catalytic cracking catalyst making up the binder of the active mass according to the invention (FIG. 2A) and the active mass according to the invention, obtained after impregnation of metal salts on said used catalyst and calcination (FIG. 2B) (example 2),

FIG. 3 shows the evolution of the grain size distribution between a used catalytic cracking catalyst making up the binder of the active mass according to the invention (FIG. 3A) and the active mass according to the invention obtained after impregnation of metal salts on said used catalyst and calcination (FIG. 3B) (example 3),

FIG. 4 shows the evolution of the relative weight loss and regain of the ilmenite sample (non-conforming) as a function of time for 5 successive reduction/oxidation cycles. In accordance with the protocol described in Example 4, the nature of the gases used (air, nitrogen, gaseous mixture CH₄/CO₂) and the temperature vary during the course of each cycle,

FIG. 5 shows the evolution of the relative weight loss and regain of the sample of example 1 (impregnated fresh catalytic cracking catalyst, in accordance with the invention) as a function of time for 5 successive reduction/oxidation cycles. In accordance with the protocol described in Example 4, the nature of the gases used (air, nitrogen, gaseous mixture CH₄/CO₂) and the temperature vary during the course of each cycle,

FIG. 6 shows the evolution of the relative weight loss and regain of the sample of example 2 (in accordance with the invention) as a function of time for 5 successive reduction/oxidation cycles. In accordance with the protocol described in Example 4, the nature of the gases used (air, nitrogen, gaseous mixture CH₄/CO₂) and the temperature vary during the course of each cycle,

FIG. 7 shows the evolution of the relative weight loss and regain of the sample of example 3 (in accordance with the invention) as a function of time for 5 successive reduction/oxidation cycles. In accordance with the protocol described in Example 4, the nature of the gases used (air, nitrogen, gaseous mixture CH₄/CO₂) and the temperature vary during the course of each cycle.

### EXAMPLES

The examples below illustrate the invention by way of non limitative example.

### Example 1

### Not Industrially Used FCC Catalyst

A not industrially used (fresh) FCC catalyst having a BET surface area of 220 m²/g and an initial pore volume of 0.8 ml/g is dry impregnated with an iron nitrate solution containing 13.9 mass % Fe₂O₃ equivalent. After calcination in air at 600° C., the impregnated catalyst contains 12 mass % iron oxide. The impregnation/drying/calcination operations are repeated three times, the active mass particles obtained having a Fe₂O₃ total mass content of 32%.

### Example 2

### Weakly Metal-Laden Industrially Used FCC Catalyst

A used FCC catalyst from an industrial unit, containing 4000 ppm nickel (Ni) and 2000 ppm vanadium (V), with a BET surface area of 107 m²/g and an initial pore volume of 0.67 ml/g, is dry impregnated with an iron nitrate solution containing 13.9 mass % Fe₂O₃ equivalent. After calcination in air at 600° C., the impregnated catalyst contains 11 mass % iron oxide. The impregnation/drying/calcination operations are repeated three times, the active mass particles obtained having a Fe₂O₃ total mass content of 30%.

### Example 3

### Heavily Metal-Laden Industrially Used FCC Catalyst

An industrially used FCC catalyst from an industrial unit, containing no nickel but 100 ppm vanadium (V), with a BET surface area of 192 m²/g and an initial pore volume of 0.64 ml/g, is dry impregnated with an iron nitrate solution containing 13.9 mass % Fe₂O₃ equivalent. After calcination in air at 600° C., the impregnated catalyst contains 12 mass % iron oxide. The impregnation/drying/calcination operations are repeated three times, the active mass particles obtained having a Fe₂O₃ total mass content of 33%.

Size Distribution of the Particles

The size distribution of the particles has been measured by wet laser grain size analysis, and the results are shown in the table hereunder.

| | Example 1 | | | Example 2 | | | Example 3 | | |
|---|---|---|---|---|---|---|---|---|---|
| | D_{V10} | D_{V50} | D_{V90} | D_{V10} | D_{V50} | D_{V90} | D_{V10} | D_{V50} | D_{V90} |
| Before impregnation | 39 | 78 | 149 | 38 | 71 | 129 | 32 | 61 | 111 |
| after impregnation/ | 7 | 62 | 142 | 19 | 73 | 140 | 4 | 63 | 126 |
| calcination | | | | | | | | | |

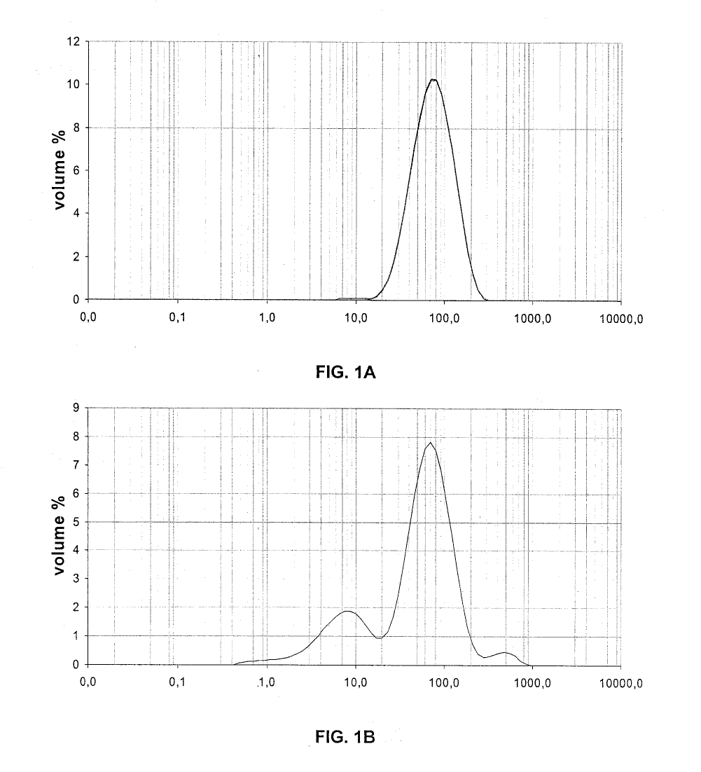

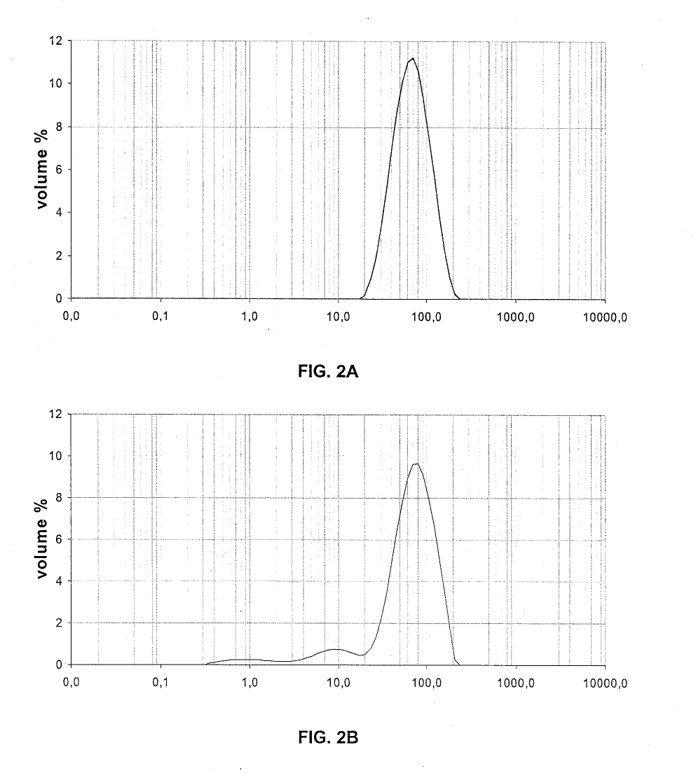

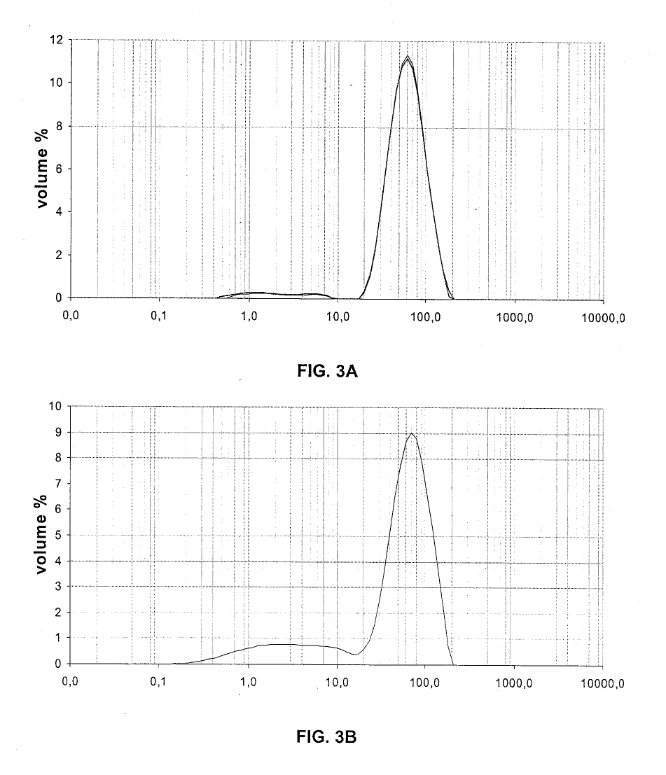

FIGS. 1, 2 and 3 respectively show the size distribution of the particles of examples 1, 2 and 3.

After dry impregnation and calcination, the size distribution of the particles is similar to the initial distribution, which allows to use FCC catalyst particles in a circulating fluidized-bed combustion process without any additional shaping stage.

Attrition Measurements

An attrition test ASTM No. D5757-00 simulating the attrition of the particles in a fluidized bed was carried out on the samples before and after impregnation/calcination. Impregnation and calcination slightly decrease the attrition resistance of the particles. The mechanical strength of the impregnated/calcined particles is suited for use in a circulating fluidized bed.

The table hereunder shows the inventory losses, in mass %, associated with attrition in the IFP standard test.

| | Example 1 | Example 2 | Example 3 |
|---|---|---|---|
| before impregnation | 2.6% | 3.7% | 1.3% |
| after impregnation/ | 2.8% | 3.6% | 5.5% |
| calcination | | | |

### Example 4

### Reactivity

A SETARAM thermobalance has been equipped with a gas supply automaton allowing to simulate the successive reduction/oxidation and oxidation stages to which the particles are subjected in a looping redox method on active mass of Chemical Looping Combustion type.

The tests are carried out at a temperature of 900° C., with 65 mg (±2 mg) sample contained in a platinum boat. In order to allow comparison between the various samples, the size distribution of the particles is selected between 30 and 40 μm by sieving. The reduction gas used is made up of 10% CH₄, 25% CO₂ and 65% N₂, and the oxidation gas is dry air.

For safety reasons, the ovens of the thermobalance are systematically subjected to nitrogen sweep between the oxidation and reduction stages.

For each sample, five, successive reduction/oxidation cycles are carried out according to the following protocol:

1) Temperature rise under air (50 ml/min):

From 20° C. to 800° C.: 40° C./min

From 800° C. to 900° C.: 5° C./min

2) Nitrogen sweep for 5 min 15 s, flow rate 80 ml/min

3) Injection of a CH₄/CO₂ mixture for 20 min, at 50 ml/min

4) Nitrogen sweep 5 min 15 s

5) Air injection, 20 min, 50 ml/min.

Stages 2 to 5 are then repeated four more times at 900° C.

| | Oxygen transfer | Reduction rate | Oxidation rate |
|---|---|---|---|
| | capacity (%) | (mmol O₂/min · g) | (mmol O₂/min · g) |
| Example 1 | 2.6 | 0.37 ± 0.02 | 0.54 ± 0.03 |
| Example 2 | 2.7 | 0.40 ± 0.02 | 0.55 ± 0.03 |
| Example 3 | 3.0 | 0.40 ± 0.02 | 0.64 ± 0.03 |
| Ilmenite | 4.1 | 0.19 ± 0.02 | 1.08 ± 0.06 |

The reduction and oxidation rates are calculated from the slopes linked with the mass loss and gain (respectively) observed, between the second and the third minute after passage under the reducing gas, and averaged over the last four redox cycles.

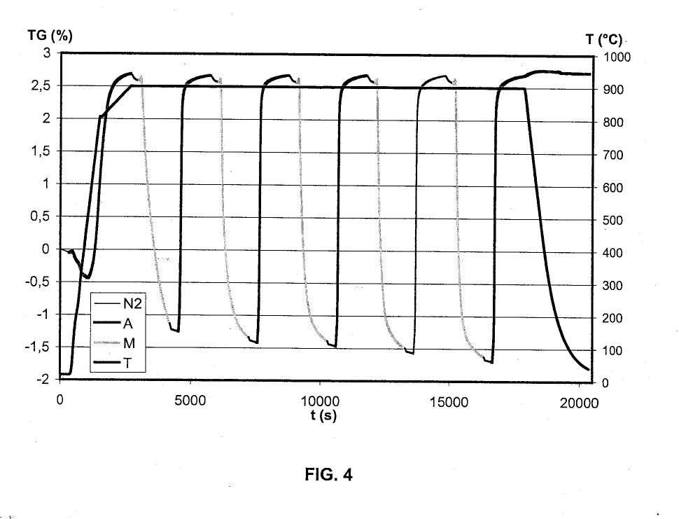

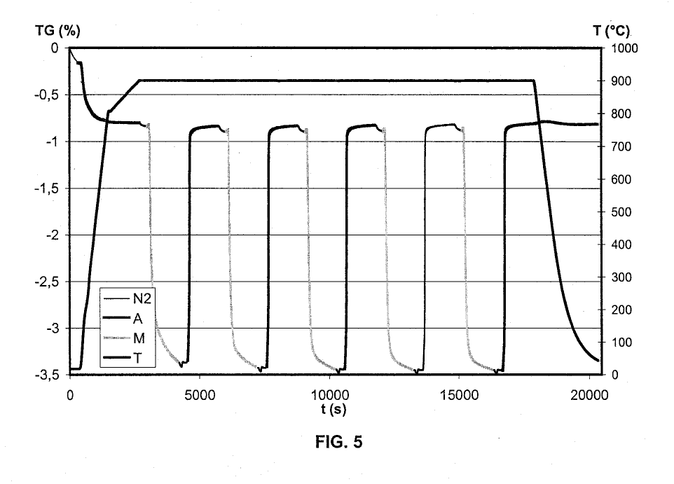

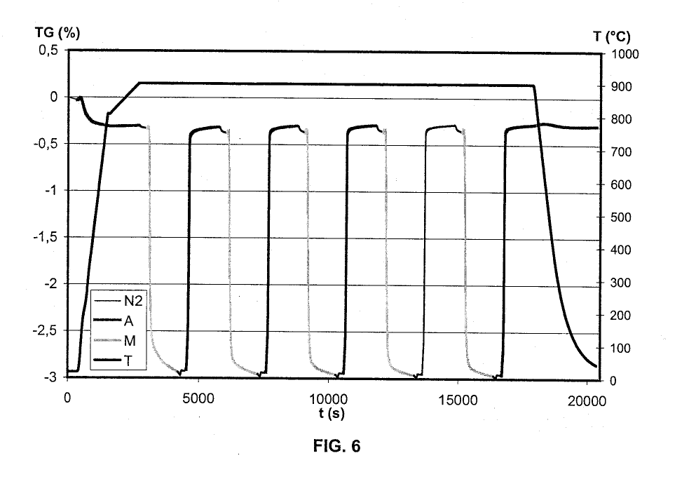

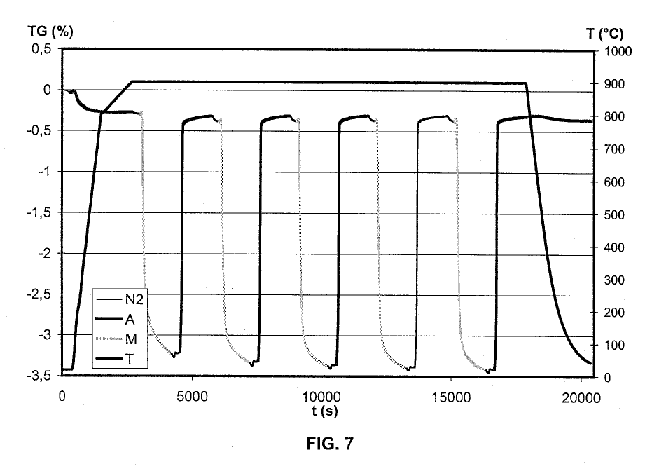

The results obtained with an ilmenite sample and the active mass samples of examples 1 to 3 are shown in FIGS. 4 to 7. These figures show the evolution of the relative weight loss and regain of the sample as a function of time for five successive reduction/oxidation cycles. In accordance with the protocol described above, the nature of the gases used varies during the course of each cycle.

The reduction rates measured by the thermobalance on the particles according to the invention are similar for the three examples and higher than the rate observed with the ilmenite. The measured oxidation rates are lower with the particles according to the invention than with the ilmenite. Similarly, the oxygen transfer capacity of the materials according to the invention is lower than for the ilmenite, but the same transfer capacity can be reached by impregnating more metals.

The comparison with ilmenite is interesting because this material is a natural oxide available on a large scale and at a relatively low cost, whose use can be considered on a large scale for coal combustion by circulating fluidized-bed CLC, the goal being to minimize the cost of the metal oxide in the process. It can thus be seen that the use of a fresh catalytic cracking catalyst and the use of a used catalytic cracking catalyst, initially considered to be a refining industry waste, allows to obtain similar performances, also at low cost.

1. A combustion method for solid, liquid or gaseous hydrocarbons through chemical looping oxidation-reduction using an active mass comprising at least one silica and alumina based binder in form of a fluidized-bed catalytic cracking catalyst and at least one metal oxide in a proportion ranging between 5 and 95 mass %.

2. A method as claimed in claim 1, wherein the binder is a used fluidized-bed catalytic cracking catalyst.

3. A method as claimed in claim 1, wherein the metal oxide content ranges between 20 and 70 mass %.

4. A method as claimed in claim 3, wherein the metal oxide content ranges between 30 and 60 mass %.

5. A method as claimed in claim 1, wherein the metal oxide is based on at least one element selected from among Co, Fe, Mn, Cu, Ni.

6. A method as claimed in claim 5, wherein the metal oxide is based on Fe.

7. A method as claimed in claim 1, wherein the combustion is total.

8. A method as claimed in claim 1, wherein the combustion is partial.

9. A method of producing synthesis gas (CO+H₂) as claimed in claim 8.

10. A method as claimed in claim 8, wherein the gas allowing fluidization of the active mass comprises steam and wherein a gaseous mixture comprising (CO₂+H₂) is produced at the outlet.

11. A method as claimed in claim 1 for energy production.

12. A method as claimed in claim 10 for hydrogen production.

13. A method as claimed in claim 9 for Fischer-Tropsch synthesis of liquid hydrocarbons from synthesis gas (CO+H₂).

14. A method as claimed in claim 7 for CO₂ capture.

15. A method as claimed in claim 1, wherein the active mass is prepared by:
a. a stage of impregnation by at least one metal salt of a fluidized-bed catalytic cracking catalyst,
b. a stage of drying and/or calcination of the impregnated catalyst.

16. A method as claimed in claim 15, wherein the active mass is:
b. dried,
c. injected after drying in the oxidation reactor.

17. A method as claimed in claim 15, wherein the active mass is:
b. calcined,
c. injected after calcination in the reduction oven.
